(19) United States
(12) Patent Application Publication
(10) Pub. No.: US 2002/0025583 A1
(43) Pub. Date: Feb. 28, 2002

(54) ANALYTICAL ROTOR AND METHOD FOR DETECTING ANALYTES IN LIQUID SAMPLES

(76) Inventors: STOUGHTON L. ELLSWORTH, PALO ALTO, CA (US); ARJUNA R. KARUNARATNE, FREMONT, CA (US); JEFFREY A. PIERCE, PALO ALTO, CA (US); LAWRENCE M. ENSLER, LOS ALTOS, CA (US); ROBERT F. ZUK, BURLINGAME, CA (US); ERIC K. GUSTAFSON, PALO ALTO, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 08/521,615

(22) Filed: Aug. 31, 1995

(51) Int. Cl. G01N 33/558

(52) U.S. Cl. 436/514

(57) ABSTRACT

An analytical rotor intended primarily for performing immunoassays comprises one or more inlet chambers for sample, wash reagents, and labelling reagents. A reaction chamber is disposed radially outwardly from the inlet chambers and connected thereto by low flow resistance flow paths. A collection chamber is located radially outwardly from the reaction chamber and connected thereto by a high flow resistance flow path. Samples are introduced to the sample inlet chamber by a transfer device, with sample volumes optionally determined by detecting when the sample inlet chamber is filled. Reagents initially introduced to the inlet chambers may be selectively transferred to the reaction chamber by low speed rotation of the rotor. The reaction chamber may then be emptied by high speed rotation of the rotor. In this way, heterogeneous immunoassays requiring sequential contact of reaction zones with sample and different reagents may be performed.

The subject matter of the present application is related to that disclosed in each of the following U.S. patent applications which are being filed on the same day: Ser. No. ______ (attorney docket no. 16415-001300); Ser. No. ______ (attorney docket no. 16415-001400); Ser. No. ______ (attorney docket no. 16415-001500); and Ser. No. (attorney docket no 16415-001700), the full disclosures of which are incorporated herein by reference.

### BACKGROUND OF THE INVENTION

1. Field of the Invention

The present invention relates generally to apparatus and methods for the detection of analytes in liquid samples. More particularly, the present invention relates to an analytical rotor and method for the immunological detection of analytes in plasma and other biological samples.

A variety of automated analytical systems have been developed for the detection and measurement of biological and other analytes in liquid samples. While such systems can be classified in many ways, the present invention is particularly concerned with two categories of analytical techniques.

The first category is generally referred to as immunological detection or “immunoassays.” Such immunological methods generally rely on the ability of antibodies and other biological receptors and ligands to specifically recognize the presence of a particular analyte in a liquid sample. While a large number of particular formats exist for detecting such binding and correlating such binding with the presence and/or amount of analyte in the sample, most such protocols rely on modulating a detectable signal based on the amount of analyte originally present in the sample. Exemplary signals include color (which can be spectrophotometrically detected), fluorescence, luminescence, radioactivity, and the like.

The second category of analytical techniques which is relevant to the present invention comprises the use of analytical rotors for performing some or all the steps necessary for the testing protocol. Usually, rotation of the analytical rotor is relied on to transfer liquid sample and other liquid reagents between various reaction and detection chambers, mixing of the liquid sample with reagents and diluents, and the like. Analytical rotors can be advantageous in that they provide a self-contained platform for performing the desired analytical methods. In particular, the use of an analytical rotor is often relied on for the separation of cellular components from whole blood to produce plasma suitable for testing.

Heretofore, analytical rotors have been most widely used for performing enzymatic and other non-immunological testing procedures. Because of their convenience, and in particular because they afford substantially complete containment of blood, plasma, and other potentially hazardous biological materials, it would be desirable to provide rotors which are useful for performing immunological detection methods. More specifically, it would be desirable to provide analytical rotors which are inexpensive to produce and which contain no or few moving parts. In particular, such rotors should provide for the sequential passage of liquid sample, diluents, washes, and other reagents necessary for performing the immunoassay past a solid phase reaction zone where the immunological reactions underlying the assay protocol would take place. More preferably, the analytical rotor and method would provide for the simultaneous assay of multiple analytes, most preferably in the form of a panel of analytes useful in performing particular diagnoses.

2. Description of the Background Art

U.S. Pat. No. 4,314,968, describes an analytical rotor intended for performing immunoassays. Analytical rotors intended for separating cellular components from whole blood samples and distributing plasma to one or more peripheral cuvettes are described in U.S. Pat. Nos. 3,864,089; 3,899,296; 3,901,658; 4,740,472; 4,788,154; 5,186,844; and 5,242,606. Analytical rotors intended for receiving sample liquids and transferring the samples radially outward by rotation of the rotor, usually with dilution of the sample, are described in U.S. Pat. Nos. 3,873,217; 4,225,558; 4,279,862; 4,284,602; 4,876,203; and 4,894,204.

### SUMMARY OF THE INVENTION

The present invention provides apparatus and methods which permit the use of analytical rotor technology for performing immunological analysis of liquid samples. In particular, the present invention provides for a relatively simple rotor construction which can be fabricated at a very low cost and which does not require the incorporation of moving parts for effecting sample and reagent flow control as the rotor is rotated (although mixing balls and other movable components could be included in order to provide other capabilities). As a particular advantage, flow control within the rotor is achieved by utilizing different rotational rates to selectively effect liquid transfer between chambers. Although particularly intended for immunological analytical protocols, the rotor of the present invention can be used for enzymatic and other non-immunological procedures as well.

An analytical rotor constructed in accordance with the principles of the present invention comprises a rotor body having a coupling element which defines an axis of rotation. The coupling element is typically a receptacle for receiving the spindle of a rotor, but could be any device or mechanism which permits detachable mounting of the rotor on a centrifugal drive unit. The rotor body includes an inlet chamber having a sample application port which permits introduction of liquid sample from an external dispenser. The rotor body further includes a reaction chamber disposed radially outwardly from the inlet chamber and a collection chamber disposed radially outward from the reaction chamber. Flow control among the chambers is achieved by connecting the reaction chamber to receive liquid flow from the inlet chamber by a first flow path in the rotor body, where the first flow path has a flow resistance selected to pass liquid at a first rate of rotation of the rotor body, typically being a low rate in the range of 100 rpm to 1000 rpm, usually 300 rpm to 900 rpm. The collection chamber is positioned to receive liquid flow from the reaction chamber by a second flow path having a much higher flow resistance selected to substantially inhibit liquid flow at the first rate of rotation. Flow through the second flow path (and emptying of the reaction chamber to the collection chamber) can be achieved at a second rate of rotation greater than the first rate of rotation, typically by a factor of at least about four. Usually, the first flow path will have a relatively large cross-sectional area, typically being greater than 0.5 mm², and a relatively short length, typically being less than 5 mm. In contrast, the second flow path has a relatively small cross-sectional area, typically less than 0.1 mm², and a much greater length, typically more than 25 mm. Additionally, the second flow path can be directed along a spiral or other non-direct (i.e., nonradial) path from the reaction chamber to the collection chamber to further enhance resistance. In this way, substantially no overflow from the reaction chamber to the collection chamber occurs during the first rotation at the first rotational speed, while the reaction chamber can be quickly emptied by the second rotation at the much higher rotational rate.

In the exemplary embodiment, the analytical rotor further includes a wash chamber disposed radially inwardly from the reaction chamber, where the wash chamber has a wash application port and is connected to the reaction chamber by a third flow path having a flow resistance selected to pass wash liquid at the first rate of rotation. The exemplary analytical rotor also includes a label chamber disposed radially inwardly from the reaction chamber. The label chamber includes a label application port (which may receive a label-containing fluid, or which may receive a fluid which does not contain label but which rehydrates dry label reagent within the label chamber) and is connected to the reaction chamber by a fourth flow path having a flow resistance selected to pass labelling reagent liquid at the first rate of rotation. In this way, plasma, wash liquids, and labels and other reagents can be selectively introduced from their respective chambers into the reaction chamber without significant overflow or loss of these liquids into the collection chamber. Each of these liquids, however, can be readily and selectively transferred into the collection chamber simply by rotating the rotor at the second rotational rate.

Usually, the reaction chamber will include at least one discrete reaction zone comprising an immobilized specific binding substance on a wall or other solid phase therein. Usually, the reaction chamber will include at least two discrete reaction zones, and more usually will include three or more discrete reaction zones. In this way, multiple analytes can be detected simultaneously in small volumes of patient plasma.

In a specific aspect of the present invention, a vapor collection region will be provided within the reaction chamber. The vapor collection region will be spaced radially inward from the reaction zones(s) and will preferably have a depressed “lower” surface so that air and other gases present in the chamber will move to this region as the rotor is rotated at the first rate of speed. In this way, the liquid sample and other reagents will cover the reaction zone(s) without discontinuities caused by vapor pockets. The region is preferably disposed at the innermost end of a radially tapered inward wall.

In another specific aspect of the apparatus of the present invention, at least a portion of the inner walls of the chambers and flow paths of the analytical rotor will be hydrophobic. In particular, hydrophobic wall portions within the reaction chamber may enhance the rate of protein binding (via adsorption) and decrease the desorption of proteins during the assay protocols. More importantly, hydrophobic surfaces within the flow paths further decrease the likelihood of overflow and fluid capillary action which might cause accidental fluid transfer when the rotor is not being rotated. That is, the hydrophobic surfaces greatly decrease the likelihood that liquid would enter any of the flow paths in the absence of outwardly radial forces generated by the rotation of the rotor body. Further, hydrophobic surfaces within the sample application chamber facilitate the movement, venting, and collection of air during liquid filling and transfer operations.

According to the method of the present invention, a measured amount of a liquid sample is applied to an inlet chamber of the analytical rotor. The rotor is initially rotated at a first rate of rotation to transfer liquid sample from the inlet chamber to a reaction chamber. The reaction chamber includes a binding substance specific for the analyte immobilized in at least one reaction zone therein. After permitting binding to occur between the binding substance and analyte (if present within the sample), the sample is transferred to a radially outward collection chamber in the rotor by rotating the rotor at second rate higher than the first rotational rate. The presence or amount of analyte in the sample can then be detected based on signal mediated by the analyte which has been competitively or non-competitively bound to the binding substance within the reaction chamber.

Usually, the reaction chamber will contain a plurality of binding substances specific for different analytes. The detection protocol will normally comprise introducing a label to the reaction chamber where the label specifically binds to analyte previously captured by the immobilized binding substance. The label can then be detected to determine the amount of analyte present in the sample.

The rotor and method of the present invention provide particularly advantageous techniques for filling the inlet chambers with sample, diluents, and other reagents. The inlet chambers may be precisely dimensioned so that, when filled to a predetermined level or point, there is an exact quantity of liquid transferred to the rotor. The use of hydrophobic surfaces, as described above, further assures that the chamber(s) will be completely filled. Filling may be accomplished using a transfer pump and a fill detection apparatus, such as a refractive index detector. When fluid is filled to any predefined point, usually a location within the low resistance flow path connecting the inlet chamber to the reaction chamber, flow is immediately stopped. Alternatively, the rotor and methods of the present invention could rely on the transfer of premeasured quantities of sample and/or other reagents, in which case the volume of the inlet chamber(s) would be less critical.

### BRIEF DESCRIPTION OF THE DRAWINGS

FIG. 1 is a top plan view of an analytical rotor constructed in accordance with the principles of the present invention.

FIG. 2 is an enlarged, detailed view of a portion of the analytical rotor of FIG. 1 showing the relative locations of the reaction zones within the reaction chamber.

FIG. 2A is a cross-sectional view taken along line 2A-2B on FIG. 2.

FIG. 3 is a schematic illustration of a system for detecting an analyte using the analytical rotor of FIG. 1.

FIG. 3A illustrates an optional sub-system which may be employed with the system of FIG. 3, where the level of fluid filling within an inlet chamber is detected based on a change in optical refractance.

FIG. 3B illustrates a platform having permanent magnets fixed therein for mixing magnetic balls in a labelling or other chamber in the rotor as the rotor is spun.

FIG. 3C is a schematic illustration of the platform of FIG. 3B and a rotor having magnetic mixing balls.

FIGS. 4A-4I illustrate the movement of liquid sample, wash fluid, and labeling reagent through the chambers of the analytical rotor of FIG. 1 during performance of an exemplary immunoassay protocol.

### DESCRIPTION OF THE SPECIFIC EMBODIMENTS

The present invention provides apparatus and methods for analyzing liquid samples, particularly biological fluids such as plasma, urine, sputum, semen, saliva, ocular lens fluid, cerebral fluid, spinal fluid, amniotic fluid, and tissue culture media, as well as food and other complex organic substances. The present invention is particularly suitable for performing immunoassay, where the target analyte may be any molecule, compound, or other substance which is suspected of being present in the sample. The target substances will usually be biological molecule, such as a polypeptide, protein, carbohydrate, or nucleic acid, and will be associated with a particular biological, pharmacological, genetic, or biochemical property of interest.

The target analytes will be detected through binding to a “specific binding substance” which is defined herein as a macromolecular compound having spacial and polar features which permit it to bind specifically to the target analyte. Specific binding substances useful in the present invention will be selected or prepared to specifically bind to particular compositions such as the target analyte. Natural specific binding pairs include antigens and antibodies, haptens and antibodies, lectins and carbohydrates, hormones and hormone receptors, enzymes and enzyme substrates, biotin and avidin, vitamins and vitamin binding proteins, complimentary polynucleotide sequences, drugs and receptors, enzymes and reaction products, enzymes and inhibitors, apoproteins and cofactors, immunoglobulins and receptors, organisms and receptors, growth factors and receptors, chelating agents and metals, and the like. Biotin and avidin derivatives may also be used, including biotin analogs/avidin, biotin/streptavidin, and biotin analogs/streptavidin. Where no natural specific binding substance exists, one may be prepared. For antigenic and haptenic target substances, antibodies may be prepared by well-known techniques. For polynucleotides, complimentary DNA or RNA fragments may also be prepared by well-known synthesis techniques.

The present invention employs an analytical rotor to receive, manipulate, and analyze the liquid sample in such a way that the presence and/or amount of the target analyte therein can be determined. The analytical rotor comprises a rotor body which is capable of being mounted on a conventional or specialized laboratory centrifuge. Conventional laboratory centrifuges are commercially available from suppliers, such as Beckman Instruments, Inc., Spinco Division, Fullerton, Calif.; Fischer Scientific, Pittsburgh, Pa.; VWR Scientific, San Francisco, Calif., and others. Generally, the rotor body of the present invention will include a receptacle or other coupling device suitable for mounting on a vertical drive shaft within the centrifuge. The particular design of the receptacle and coupling device will depend on the nature of the centrifuge, and it will appreciated that the rotor body of the present invention may be adapted to be used with many types of centrifuges which are now available or which may become available in the future.

The rotor body of the present invention comprises a body structure which maintains a desired geometric pattern or relationship between a plurality of chambers and flow paths, as described in more detail hereinbelow. Usually, the body will be a solid matrix with the chambers and passages formed therein as spaces or voids. Conveniently, the rotor bodies of the present invention may be formed by laminating a bottom portion having the chambers and flow passages formed therein, typically by conventional molding processes, and a top portion or cover which may be laminated to the bottom portion by conventional techniques, such as ultrasonic welding. The final enclosed volumes are formed when the layers are brought together. Of course, the rotor body could be formed as a plurality of discrete components, such as tubes, vessels, chambers, etc., arranged in a suitable structural framework. Such assemblies, however, will generally be more difficult to manufacture and are therefore less desirable than those formed from a solid matrix.

The rotor body may be formed from a wide variety of materials, and may optionally include two or more materials. The materials should be compatible with the intended assay protocols. For example, it is generally desirable that the material be non-fluorescent, and usually that the material be capable of passing light through suitable optical paths in order to permit the contents to be observed spectrophotometrically, fluorometrically, luminescently, or by other optical assessment techniques. In the exemplary embodiment described below, the rotor is formed from polystyrene and a polyacrylate.

In a preferred aspect of the present invention, the rotor body is formed in an upper layer and a lower layer. The upper layer is formed from an optically transmissive material, such as polystyrene. The lower layer (which forms the bottom of the rotor body) is formed from an opaque material, usually black material, which reduces background fluorescence. The black material may be polyacrylate or polystyrene with a suitable black pigment or filler added.

The analytical rotor of the present invention is particularly intended for performing heterogeneous immunoassays where it is necessary to sequentially contact a reaction zone with liquid sample, wash reagents, labelling reagents, and the like, in order to bind label within the reaction zone in an amount mediated by the amount of analyte initially present in the sample. The particular order in which the various reagents are contacted with the reaction zone(s) depends on the specific protocol being employed, and a wide variety of well-known immunoassay protocols may be performed using the analytical rotor of the present invention. Heterogeneous immunoassays may generally be classified as either competitive or non-competitive.

Generally, competitive immunoassay formats provide for introducing labelled analyte or analyte analog together with the liquid sample, where the labelled analyte competes with native analyte for binding to immobilized binding substance specific for the analyte. The amount of immobilized binding substance is limited so that labelled analyte will compete with native (unlabelled) analyte for binding to the limited number of binding sites (i.e., not all of the labelled and/or unlabelled analyte can be bound within the reaction zone). In this way, the amount of label bound within the reaction zone will be inversely proportional to the amount of analyte initially present in the sample. Such protocols are generally suitable for the detection of small molecules, such as drugs and haptens, and require that the sample be mixed with the labelled analyte or analyte analog prior to introduction to the reaction zone. With the methods of the present invention, such mixing could occur in the analytical rotor (e.g., by simultaneously or sequentially introducing the sample and the reagent containing labelled analyte to a chamber and mixing prior to passing the combined solution to a reaction zone). Alternatively, mixing could occur prior to introducing the combined sample/labelled analyte solution to the analytical rotor.

The analytical rotor and methods of the present invention are particularly suitable for performing non-competitive (sandwich) assay formats where sample is first introduced to a reaction zone having an excess amount of binding substance specific for the analyte (i.e., sufficient binding substances present to assure binding of all analyte which may be present in the sample). After washing the reaction zone, a labelling reagent will be introduced to the reaction zone, where label is bound to a binding substance specific for the analyte. After washing, the amount of label bound within the reaction zone can be determined and related to the initial concentration of analyte present in the sample.

Most competitive and non-competitive assay formats rely on immobilization of a binding substance specific for the analyte within a reaction zone. The manner in which the binding substance is bound will depend on the type of binding substance. In the case of antibodies, binding may occur either directly or indirectly, e.g., through the use of intermediate binding substances such as biotin and avidin. Immobilization of the binding substances within the reaction zone may be covalent or non-covalent, with non-covalent binding being preferred with the use of hydrophobic surfaces within the reaction zone, as described in more detail below. Particularly suitable methods for immobilizing haptens are described in copending application Ser. No. 08/374,265 (attorney docket no. 16415-000800) and for proteins are described in copending application Ser. No. ______ (attorney docket no. 16915-001700), the full disclosures of which are incorporated herein by reference.

The assays of the present invention will employ a labelling reagent comprising a labelling molecule which can be any compound, molecule, moiety, or the like, which can be bound to a specific binding substance so as to provide a detectable label on that substance. Suitable labelling molecules include, but are not limited to, fluorophores, chemiluminescent compounds, enzymes, enzyme cofactors, enzyme inhibitors, radioisotopes, scintillants, and the like. Preferably, the labelling molecule will be one which can be observed visually, e.g., a fluorophore, luminophore, scintillant, or chemiluminescer, or one which mediates the formation of a product that may be observed visually, e.g., a dye. In the preferred case of multiple reaction zones within a reaction chamber, as described below, the use of labelling molecules which provide a localized signal, i.e., one which can be detected within a fixed area within the reaction chamber, is preferred. Such labels include fluorophores, luminophores, chemiluminescers, and the like, which will emit detectable energy upon excitation with energy of a different wave length. In that way, each reaction zone within a reaction chamber may be separately excited without excitation of adjacent reaction zones. Thus, each individual reaction zone may be read without interference from other reaction zones.

The labelling molecules used in the labelling reagents in the present invention may be attached to a specific binding substance using a wide variety of conventional techniques. When the specific binding substance is a protein or polypeptide, the labelling molecule will usually be covalently attached to the specific binding substance, but indirect linkages such as through biotin-avidin binding or other cognate members of specific binding pairs may also find use. Usually, covalent binding will be effected through moieties naturally present on the polypeptide or protein, such as an antibody, including disulfide, hydroxyphenyl, amino, carboxyl indole, and other functional groups, using conventional conjugation chemistry as described in the scientific and patent literature. Alternatively, antibodies may be biotinylated by known techniques (see Wilchek and Bayer, ANAL. BIOCHEM. 171:1-32 (1988)) and linked to the specific binding substance through avidin molecules.

The analytical rotor of the present invention includes a plurality of liquid-receiving chambers which are interconnected to provide the controlled and sequential flow of liquid sample, wash reagents, and labelling reagents therethrough. In a specific aspect, the present invention controls flow between chambers by connecting the chambers with flow paths having a flow resistance which allows or inhibits liquid flow depending upon the rotational speed (e.g., acceleration or “g” force of the rotor). In the exemplary embodiment, flow paths are provided having either a low flow resistance which permits rapid radially outward flow of liquid at relatively low rotor rotation rates, e.g., in the range from 100 rpm to 1000 rpm (corresponding to 2 g to 12 g for rotors having a diameter from 2 cm to 12 cm), usually from 300 rpm to 900 rpm. High resistance flow paths are provided with a flow resistance selected to permit rapid flow of liquid between chambers at much higher rotational rates, typically in the range from 3600 rpm to 5400 rpm (corresponding to 370 g to 830 g). Of course, the interrelationship between the flow resistance of the flow path and flow rate of the liquid will depend on the liquid viscosity, alignment of the flow path relative to the radial direction (e.g., spirally oriented flow paths will be longer and display a slower flow than radially oriented flow paths), and the like. Generally, however, it will be desirable to limit the flow of liquid through the high resistance flow paths to 5% or less, preferably 1% or less, of the liquid flow through the low resistance flow paths at the low rotational rate. In this way, liquid can be transferred to intermediate chambers without substantial loss of the liquid until the chamber is to be intentionally emptied by rotation of the rotor at the higher rotational rate.

In another aspect, the present invention provides for hydrophobic surfaces within at least a portion of the liquid-receiving chambers and flow paths. Hydrophobic surfaces can be obtained either by appropriate material selection (e.g., most polystyrenes are hydrophobic) or by treatment to achieve hydrophobicity. Hydrophobicity helps control liquid flow rates by limiting any propensity toward capillary flow. Hydrophobicity can be imparted to the preferred acrylic rotor body by plasma etching with a hydrocarbon etchant. Preferably, etching is initially performed with argon (to clean the part) followed by a combination of CH₄ and CF₄. Such etching methods can also enhance protein binding and are described in detail in copending application Ser. No. ______ (attorney docket no. 16415-17), the full disclosure of which has previously been incorporated herein by reference.

In addition to such hydrophobic surface treatment, the internal surfaces of the rotor may be formed to have a pattern of small, capillary-dimensioned channels in order to help direct air flow within individual chambers. Because of the hydrophobic nature of the surface, these channels resist the intrusion of water, and thus permit the air to flow through the chamber even when the main volume of the chamber is filled with liquid. For example, such air channels may be formed within the reaction chamber to direct air flow to the vapor collection region. Additionally, such air channels may be formed to permit air flow to vent(s) formed within the rotor body.

The analytical rotor of the present invention will include at least one inlet chamber intended to receive liquid sample and optionally other liquid reagents. The inlet chamber will have an inlet port formed through the rotor body and will be vented to permit displacement of air as the liquid is introduced. The size of the inlet chamber should be sufficient to accommodate the expected amounts of liquid sample and other reagent(s) which may be introduced. Usually, separate chambers will be provided for the introduction of wash reagent, labelling reagent, and any other reagents which may be utilized in the analytical protocol. The use of separate inlet chambers for each reagent is desirable in several respects. First, the chance for cross-contamination is reduced. Second, the volumes and geometries of the chambers can be tailored to accommodate the corresponding reagent.

In a preferred aspect of the present invention, sample and other liquid input volumes will be measured by filling the associated inlet chamber to a precise location in the rotor. For example, the liquid sample may be transferred to a sample inlet chamber having a precisely defined volume where the chamber is filled substantially entirely with a small volume of overflow into the low resistance flow path which leads to the reaction chamber. The hydrophobic surface of the sample inlet chamber helps assure that the chamber is uniformly filled, with air being displaced in an even manner. The sample fluid will enter the low resistance flow path with a substantially uniform front, permitting optical or other detection of the precise moment when the front reaches a predetermined location within the flow path. In the exemplary embodiment, optical detection comprises sensing a change in the index of refraction caused by the advancing liquid front using a suitably aligned light source and detector, where the amount or nature of light detected indicates a change in refractance caused by passage of the sample fluid to the location. At that point, sample introduction to the inlet chamber can be stopped, thus providing for very accurate volumetric transfer of the sample liquid. Such volumeric transfer techniques can also be used for the diluent, wash fluid, labelling reagent, or the like, although it will generally be less critical to provide the high degree of accuracy for these other transfers.

The rotor of the present invention may further be provided with features which permit detection of errors in fluid transfer. For example, leakage may occur between a fluid transfer dispenser in the rotor, e.g., through misalignment. In such cases, the amount of the transferred fluid may remain on the surface of the rotor body and not enter the associated inlet port. Detection of such errors in transfer may be achieved by a variety of approaches. For example, an annular cup may be provided around the inlet port (i.e., a depression in the upper surface of the rotor body) to collect liquid which does not enter through the sample entry port. Optical means, such as a refractance detector, an optical density reader, or the like, may be provided for detecting the presence of such overflow. If overflow is detected, an error can be signalled and the analysis terminated.

The sample inlet ports may also be formed to enhance complete entry of the fluid into the inlet chamber upon spinning of the rotor. In particular, the inlet ports may be formed with an inverse chamfer (i.e., an increasing diameter in the direction from the upper surface of the rotor into the chamber) so that any fluid in contact with the chamfered surface will flow into the chamber when the rotor is spun.

The analytical rotor will also include a reaction chamber having at least one reaction zone therein. The reaction chamber is disposed radially outwardly from the inlet label, wash and other introductory chamber(s) and connected to said inlet and other chamber(s) by low resistance flow paths. Thus, liquid sample, wash reagents, labelling reagents, and the like, may be selectively transferred from the inlet and other chamber(s) to the reaction chamber by low speed rotation of the rotor, usually at an rpm in the range from 100 to 1000, preferably from 300 to 900.

The reaction chamber includes a vapor collection region disposed at a radially inward location. The vapor collection region will not overlap with any of the reaction zone(s). Thus, by introducing sufficient liquid sample and other reagents, coverage of the reaction zones can be assured with the vapor collected in the collection zone away from the reaction zones. The vapor collection region will preferably comprise an “overhead” space or volume formed within the reaction chamber at a radially inward portion thereof. Centrifugal force caused by spinning the rotor will cause air bubbles to migrate toward the radially inward portion of the reaction chamber. By orienting the radially inward wall of the reaction chamber so that it becomes increasingly close to the center of the rotor at one end thereof, the air bubbles will naturally migrate toward this closer end. By locating the overhead space at this end, the air bubbles will collect within the vapor collection region where they will tend to remain due to surface tension. The overhead region is formed in the upper surface of the reaction chamber. Thus, it will lie above the main volume of the reaction chamber when the rotor is in its normal orientation during use.

The analytical rotor will also include a collection chamber disposed radially outwardly from the reaction zone. The collection zone will be connected to the reaction zone by a high resistance flow path so that liquid sample and other reagents may be maintained in the reaction chamber while the rotor is being rotated at the low rotational rate. When it is desired to remove liquid sample or other reagent from the reaction chamber, the rotor is rotated at the high rotational rate, usually at from 3600 rpm to 5400 rpm, preferably from 4000 rpm to 5000 rpm, causing the liquid to flow out through the high resistance flow path to the collection chamber. Advantageously, all liquid sample and other reagents will be collected and maintained within the collection chamber, allowing the rotor to be disposed of without release of the potentially hazardous biological materials.

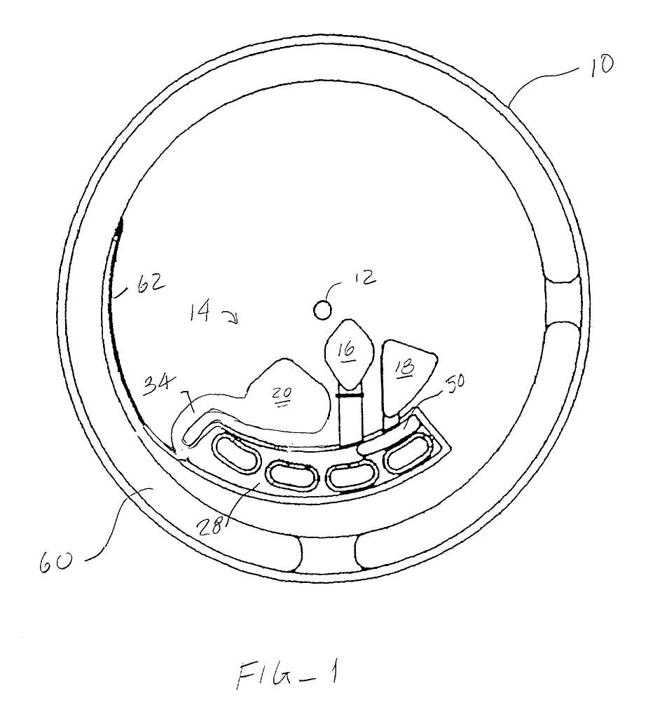

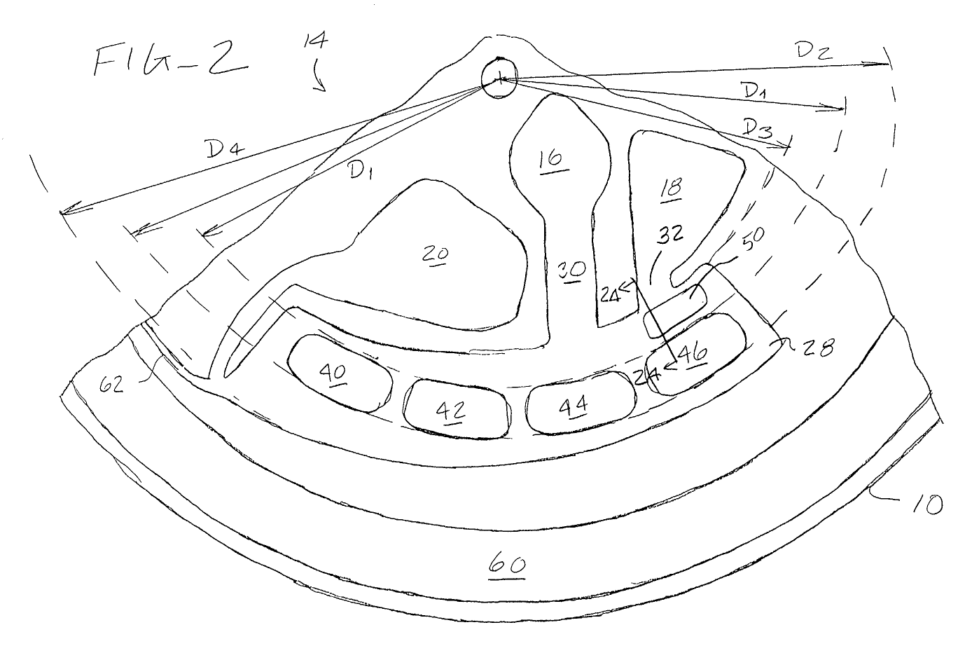

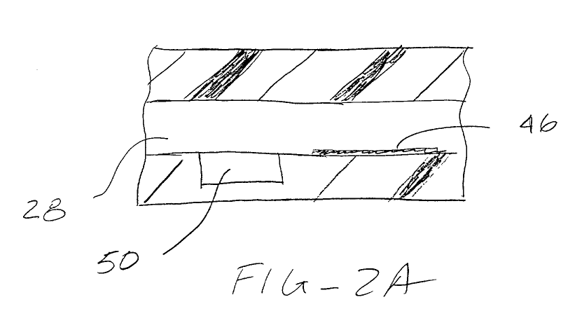

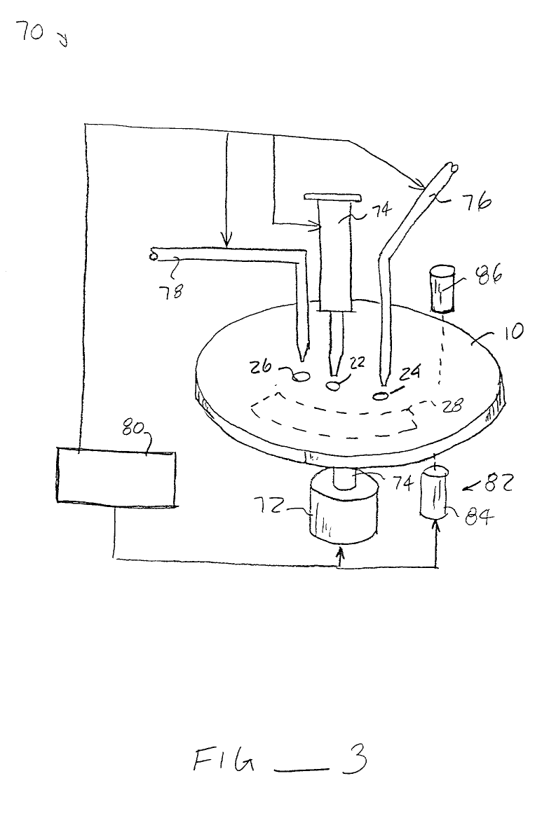

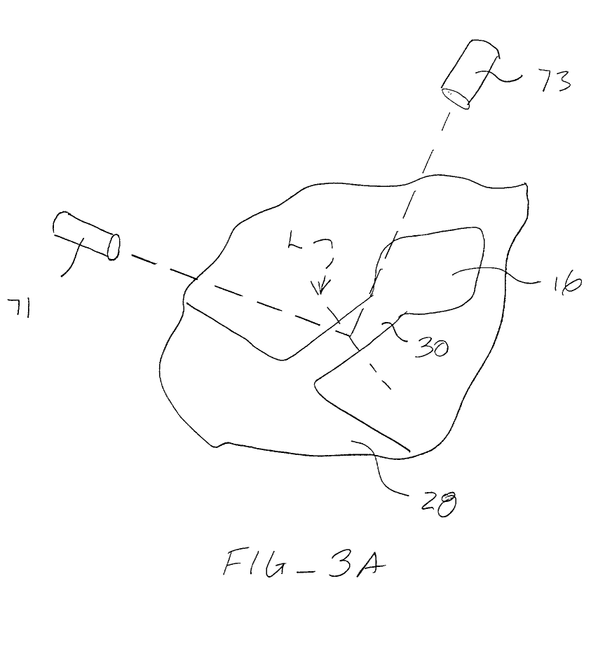

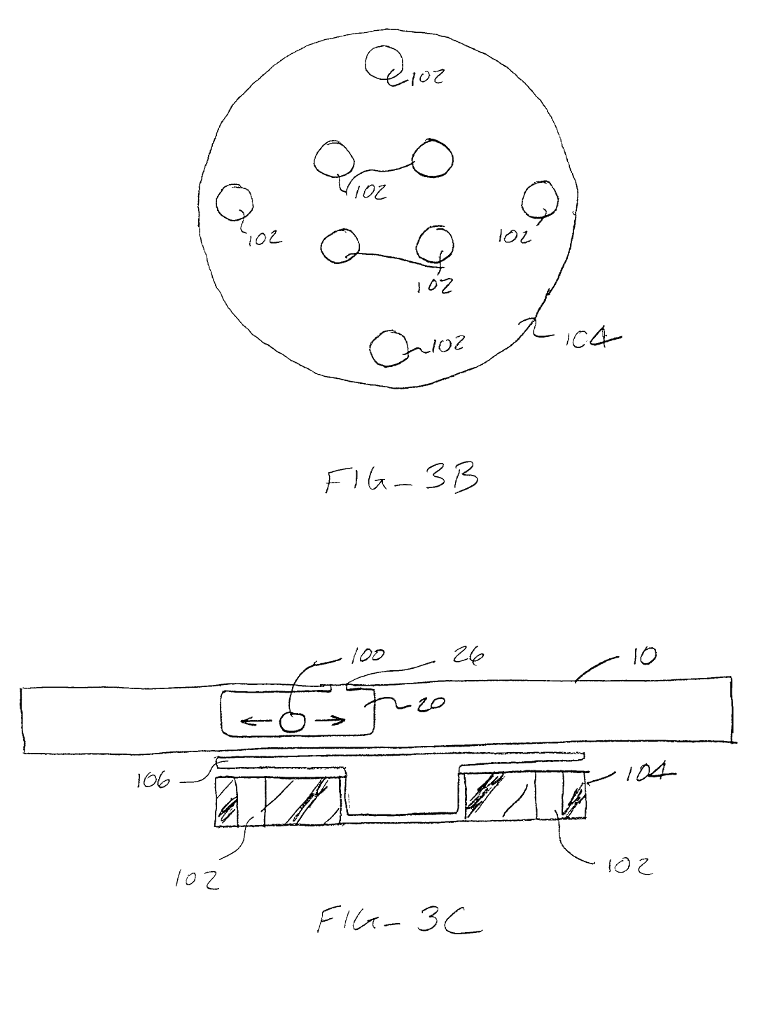

Referring now to FIGS. 1 and 2, an analytical rotor constructed in accordance with the principles of the present invention comprises a rotor body 10 which is in the form of a thin disk typically having a diameter in the range from 4 cm to 8 cm, and a thickness in the range from 4 mm to 8 mm. The rotor body 10 includes a mounting hole 12 which defines an axis of rotation and which can be placed on a spindle on a rotational drive motor, as described in more detail in connection with FIG. 3 below. As illustrated, the rotor body 10 includes a single “test panel 14” which comprises an inlet chamber 16, a wash chamber 18, and labelling reagent chamber 20. Each of the chambers 16, 18, and 20 will have an associated inlet port 22, 24, and 26, respectively (as shown in FIG. 3) to permit introduction of the appropriate liquid during performance of an assay, as described in more below. Often, it will be desirable to include two or more separate test panels on the same rotor 10.

A reaction chamber 28 is connected to each of the inlet chambers 16, 18, and 20, by connecting flow paths 30, 32, and 34, respectively. Each of the flow paths 30, 32, and 34 will have a hydrophobic surface, as described above, and in more detail in copending application Ser. No. ______ (attorney docket no. 16415-001700), the full disclosure of which is incorporated herein by reference, and will provide a sufficient barrier so that liquids initially placed into chamber 16, 18, and 20, while the rotor is stationary, will remain generally stationary and will not pass into the reaction chamber 28. Only after rotation at a speed above a threshold value, typically about 1000 rpm, will fluid in and of the chambers pass into the reaction chamber 28. It should be noted that hydrophobic surfaces are desirable in the usual case of aqueous, polar solutions. In the case of non-aqueous, non-polar fluids, it will be preferred to use a hydrophilic disk surface to inhibit fluid flow thereover.

Flow path 34 which connects the labelling reagent chamber 20 with the reaction chamber 28 is connected to the bottom (i.e., the radially outward-most point) of the reaction chamber 28. By connecting to this point of the reaction chamber 28, rather than the top (i.e., the radially inwardmost point), labelling reagent will enter the chamber from the bottom and fill upwardly during the transfer step. Such bottom delivery reduces the formation of bubbles in the reaction zone which could, in some instances, cause certain labelling reagents to foam and enter into other chambers. Such problem would be exacerbated by the possibility of trapping air bubbles within the bottom portion of the chamber, which would further displace the labelling reagent and increase the risk of the reagent entering other inlet chambers or flowing back into the labelling reagent chamber 20. Moreover, by connecting flow path 34 adjacent to the beginning of the high resistance flow path 62 (described hereinafter), the labelling reagent will be completely evacuated from the chamber 20 during the evacuation step (described hereinafter) and residue will stay far from the read zone, further reducing the risk of contaminating subsequent steps of the detection protocol with labelling reagent.

Reaction zones 40, 42, 44, and 46, will be formed within the reaction chamber 28. Usually, each of the reaction zones will be defined by immobilizing a desired specific binding substance on a geometrically defined region or pattern on a wall of the reaction chamber 28, as illustrated. Alternatively, the reaction zone(s) could be formed by attaching beads, or other structures, within the reaction chamber 28.

In a preferred aspect of the present invention, the individual reaction zones will be located within the reaction chamber so that a vapor collection region 50 is disposed in a radially inward portion of the chamber 28. As illustrated in FIG. 2, the reaction zones 40, 42, 44, and 46 may be disposed to lie within an annular region having a radially inward diameter of D1 and a radially outward diameter of D2. By positioning at least a portion of the radially inward wall of the reaction chamber 28 to a shorter radially inward diameter D3, the vapor collection region will receive and collect vapor that moves along the wall. Preferably, the vapor collection region 50 will extend downwardly (i.e., into the bottom surface of the chamber 28), as illustrated in FIG. 2A. The region which is created helps to trap and maintain air bubbles which migrate toward the collection region 50 as a result of rotation of the rotor. In particular, when the rotor is stopped, surface tension will help maintain the desired segregation between the liquid and the vapor in chamber 50. Then, so long as sufficient liquid is introduced to fill the portion of the reaction chamber which is radially outward from the diameter D1, the reaction zones will be covered by the sample and other reagent(s), while vapor will collect in the “overhead” region lying at a diameter smaller than D1.

Liquid sample and reagents may be emptied from the reaction chamber 28 to an outer collection chamber 60 through a high resistance flow path 62. The high resistance flow path 62 will be connected at a radially outward portion, typically lying at a radius D4 which is the radially outward most portion of the chamber. As illustrated, high resistance flow path 62 is much longer than the inlet flow paths 30, 32, and 34, and also has a much smaller cross-sectional area.

Referring now to FIG. 3, a system 70 for manipulating the analytical rotor of the present invention to perform an immunoassay will be described. The system 70 includes a centrifugal drive unit 72 having a spindle 74 for receiving and rotating the rotor body 10. In addition to rotating the rotor body 10 at the rotational rates described above, the drive unit 72 will be able to selectively position the stationary rotor so that the features on the rotor can be positioned relative to the liquid handling means and signal detection means of the system. The liquid handling means will include at least a sample delivery device 74, which may be of a type which filters precisely measured volumes of plasma from whole blood and dispenses the plasma through simple inlet port 22. Examples of such filtering and dispensing systems are described in detail in copending application Ser. Nos. 08/326,974, filed on Oct. 21, 1994, and 08/386,242, filed on Feb. 9, 1995, the full disclosures of which are incorporated herein by reference.

FIG. 3A illustrates a sample filling detection system, including a light source 71 and a detector 73. Light source 71 focuses a narrow beam of light so that it strikes a line L which lies at the “fill point” for the sample inlet chamber 16. The fill line L is located part way down the flow path 30 which connects the inlet chamber 16 to the reaction chamber 28. Sample is introduced through the associated sample port by conventional means, such as a fluid pump, fluid pipetter, or the like, until the sample reaches line L. At that point, the arrival of fluid causes an index of refraction change which alters the amount of light detected by the detector 73. The flow of fluid can be immediately stopped, providing for a highly accurate volumetric fluid transfer. As discussed above, the provision of a hydrophobic inner surface in the inlet chamber 16 and transfer flow path 30 helps assure that air is uniformly displaced as fluid enters the volume and the fluid does not wick prematurely, further assuring for accurate volumetric measurement.

The system 70 will further include a wash dispenser 76 which will be capable of dispensing premeasured volumes of wash fluid to the wash chamber 18 through inlet port 24. Optionally, the system 70 may include a labelling reagent dispenser 78 which is capable of dispensing premeasured volumes of a labelling reagent liquid to the labelling reagent chamber 20 through inlet port 26. Usually, however, the label will be provided in the chamber and rehydrated by introduction of a measured amount of diluent or wash fluid from dispenser 76. These dispensing means will be controlled by controller 80, which also controls the central drive unit 72 and a detection unit 82. The detection unit 82 will typically include an excitation source 84 and a detector 86. For example, the excitation source 84 may direct light or other energy at a wavelength which excites a fluorescent label within a reaction zone in the reaction chamber 28. Resulting fluorescence may then be detected by detector 86.

In order to enhance mixing of fluid in any of the chambers 16, 18, or 20, magnetic mixing balls 100 may be placed in the chamber, which is shown as the labelling chamber 20. Typically, labelling chamber 20 will have dried reagent on at least a portion of its inner surfaces. The reagent will be reconstituted upon the addition of diluent or other reagents, as discussed above. In order to enhance dissolution and reconstitution of the labelling reagent, the mixing ball 100 is provided. Mixing ball 100 is agitated by interaction with a plurality of fixed magnets 102 disposed in a platform 104 which lies beneath a rotor drive plate 106. The magnets 102 are disposed in a pattern, best illustrated in FIG. 3B, so that the magnets alternate between the radially inner side of chamber 20 and the radially outer side of chamber 20 as the rotor 10 is rotated. In this way, the magnetic ball will be caused to alternately move radially inwardly and radially outwardly as the rotor is spun.

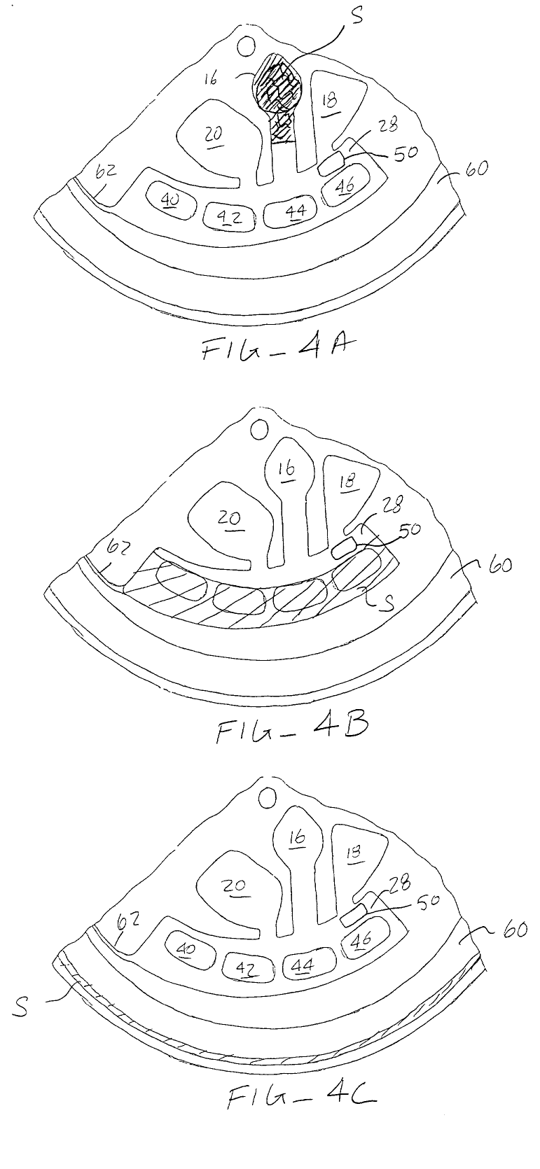

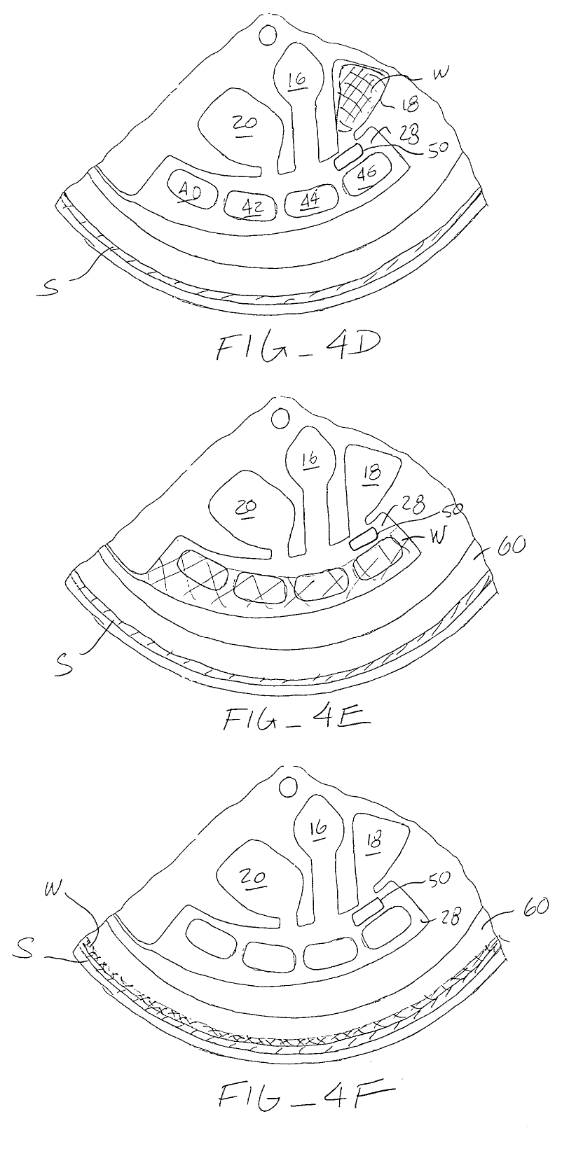

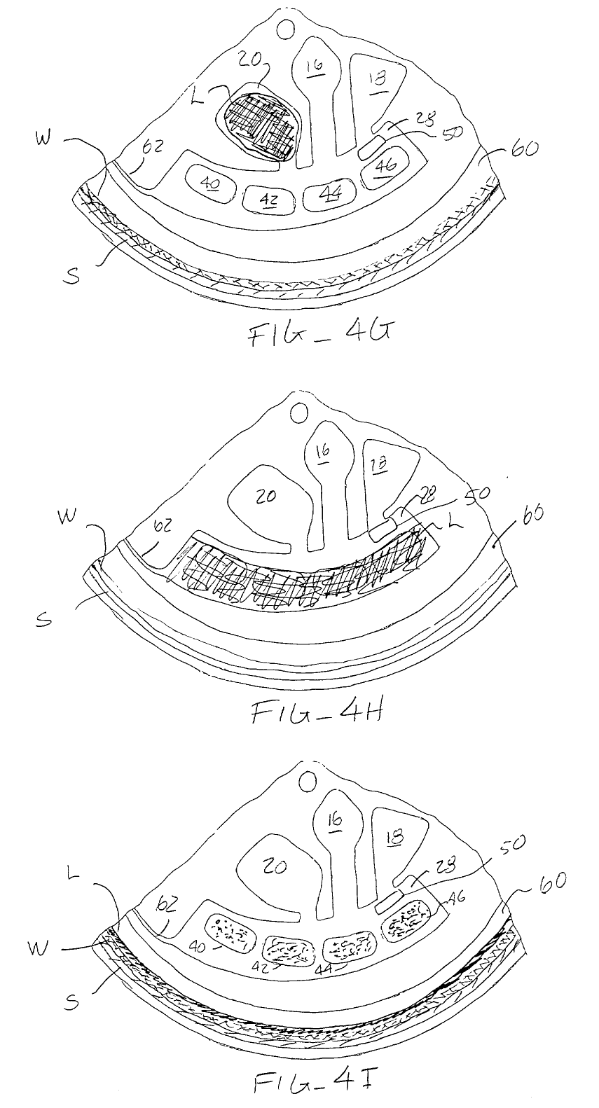

Referring now to FIGS. 4A-4I, a method according to the present invention employing the analytical rotor described above will be set forth in detail. Initially, a premeasured volume of sample S is introduced to the inlet chamber 16, as illustrated in FIG. 4A. The volume of sample may be premeasured or measured as it is delivered using the detection system of FIG. 3A. By rotation at a low rotation rate, the liquid sample S is transferred to and maintained in the reaction chamber 28, as illustrated in FIG. 4B. The initial volume of sample will be selected so that the level of sample S within the chamber 28 will extend sufficiently far in the radially inward direction so that each of reaction zones 40, 42, 44, and 46 is covered. Vapor originally in the chamber 28 will move to the vapor collection region 50 and subsequently out through chambers 18 and 20 (which may force some fluid back into chambers 18 and 20). During the initial low speed rotation, substantially no sample will be lost through the high resistance flow path 62. After the initial reaction with liquid sample S is complete, however, the sample may be removed from reaction chamber 28 and transferred to collection chamber 60 by rotating the rotor at a high rotational rate. The sample S will then lie within a peripherally outward region of chamber 60, as illustrated in 4C.

Next, a wash reagent W is introduced to the wash chamber 18, as illustrated in FIG. 4D. The wash reagent W may be sequentially transferred to the reaction chamber 28 and then to the collection chamber 60, generally as described above in connection with the sample S, and as illustrated in FIGS. 4E and 4F. Typically, the wash step will be repeated one or more times, optionally with mixing, in order to assure that non-bound analyte and other materials are removed from the reaction zones in the reaction chamber 28. At the end, all wash reagent W will be spun out through fluid path 62 and collected within the collection chamber 60 as illustrated in FIG. 4F. Optionally, small mixing balls (not shown) may be disposed within the reaction chamber 28 to enhance mixing and washing of the reaction zones.

After the reaction chamber has been washed, a pre-measured amount of labelling reagent L (which may be rehydrated from the bottom wall of chamber 20 as described above) will be introduced to the labelling chamber 20, as illustrated in FIG. 4G. The labelling reagent L is transferred to the reaction chamber 28 by low speed rotation of the rotor, as illustrated in FIG. 4H. The labelling reagent will be retained within the reaction chamber while rotating the rotor at a low rate in order to maintain coverage of all reaction zones. After the reaction is completed, the labelling reagent L will be transferred to the collection chamber 60 by rotating the rotor at the high rotational rate. The label will remain bound within the reaction zones 40, 42, 44, and 46, in an amount depending on the amount of analyte originally present in the sample and on the type of protocol which has been employed. After washing the reaction chamber 28 one or more times, generally as described above in connection with FIGS. 4D-4F, label within the reaction zones may be detected and the amount of analyte initially present in the sample determined.

Although the foregoing invention has been described in some detail by way of illustration and example, for purposes of clarity of understanding, it will be obvious that certain changes and modifications may be practiced within the scope of the appended claims.

What is claimed is:

1. An analytical rotor for performing analysis of a liquid sample, said rotor comprising:
a rotor body having a coupling element defining an axis of rotation;
an inlet chamber having a sample application port in the rotor body;
a reaction chamber disposed radially outwardly from the inlet chamber; and
a collection chamber disposed radially outwardly from the reaction chamber;
wherein the reaction chamber is connected to receive liquid flow from the inlet chamber by a first flow path having a flow resistance selected to pass liquid at a first rate of rotation of the rotor body and wherein the collection chamber is connected to receive liquid flow from the reaction chamber by a second flow path having a flow resistance selected to inhibit liquid flow at the first rate of rotation and to pass liquid flow at a second rate of rotation greater than the first rate of rotation.

2. An analytical rotor as in claim 1, wherein the first flow path has a cross-sectional area greater than 0.5 mm² length less than 5 mm, while the second flow path has a cross-sectional area less than 0.1 mm² and length greater than 25 mm.

3. An analytical rotor as in claim 1, further comprising a wash chamber disposed radially inwardly from the reaction chamber, wherein said wash chamber has a wash application port and is connected to the reaction chamber by a third flow path having a flow resistance selected to pass liquid flow at the first rate of rotation.

4. An analytical rotor as in claim 1, further comprising a label chamber disposed radially inwardly from the reaction chamber, wherein said label chamber has a label application port and is connected to the reaction chamber by a fourth flow path having a flow resistance selected to pass liquid flow at the first rate of rotation.

5. An analytical rotor as in claim 4, wherein the fourth flow path is connected to the reaction chamber near the radially outward most point on said reaction chamber.

6. An analytical rotor as in claim 1, wherein at least one specific binding substance is immobilized in a reaction zone in the reaction chamber.

7. An analytical rotor as in claim 6, wherein at least two different binding substances are immobilized in separate reaction zones within the reaction chamber.

8. An analytical rotor as in claim 7, wherein at least two specific binding substances are selected from the group consisting of anti-CKMB and anti-CKMM.

9. An analytical rotor as in claim 6, wherein the reaction chamber has a radially inward wall having a peripheral geometry which defines a vapor collection region.

10. An analytical rotor as in claim 9, wherein the vapor collection region lies radially inwardly from the reaction zone and includes a space for maintaining the collected vapor.

11. An analytical rotor as in claim 1, wherein at least a portion of the inner surfaces of the inlet chamber, reaction chamber, collection chamber, first flow path, and second flow path is hydrophobic.

12. An analytical rotor as in claim 11, wherein the rotor body is molded from a polymeric material and wherein said hydrophobic portion of the inner surfaces is formed by post-molding treatment of the surface.

13. An analytical rotor as in claim 12, wherein the surface is treated by plasma etching.

14. An analytical rotor as in claim 11, wherein the entire surface area of the in surfaces is hydrophobic.

15. An analytical rotor as in claim 11, wherein at least an inner surface of the reaction chamber is hydrophobic and wherein a specific binding protein is immobilized over said portion.

16. A method for detecting an analyte in a sample, said method comprising:
applying liquid sample to an inlet chamber in an analytical rotor;
rotating the rotor at a first rate of rotation to transfer the liquid sample from the inlet chamber to a reaction chamber having a binding substance specific for the analyte immobilized in a reaction zone therein;
rotating the rotor at a second rate of rotation higher than the first rate to transfer the liquid sample from the reaction chamber to a collection chamber; and
detecting the presence or amount of analyte in the sample based on a signal mediated by the amount of analyte competitively or non-competitively bound to the binding substance between said first and second rotating steps.

17. A method as in claim 16, wherein the specific binding substance captures analyte within the reaction chamber, and wherein the detecting step comprises attaching label to the captured analyte and measuring the amount of label attached to said analyte.

18. A method as in claim 17, wherein a plurality of binding substances specific for different analytes are immobilized within the reaction zone, wherein each of said analytes may be detected simultaneously.

19. A method as in claim 16, wherein the first rotational rate is in the range from 100 rpm to 1000 rpm and wherein the second rotational rate is in the range from 3600 rpm to 5400 rpm.

20. A method as in claim 16, wherein at least a portion of the flow surfaces within the rotor is hydrophobic.

21. A method as in claim 16, wherein the reaction chamber includes a radially inward vapor collection region which collects vapor and maintains the vapor away from the reaction zone.

22. A method as in claim 16, further comprising detecting when the sample inlet chamber is filled and stopping applying the liquid sample when filling is achieved, whereby the volume of applied liquid sample equals the inlet chamber volume.

23. A method as in claim 16, wherein a premeasured volume of sample is applied to the inlet chamber.

24. A method as in claim 16, wherein liquid within the inlet chamber is mixed by the action of a magnetic mixing ball which interacts with a plurality of fixedly disposed permanent magnets as the rotor is rotated.
